(19) United States
(12) Patent Application Publication
(10) Pub. No.: US 2004/0082996 A1
(43) Pub. Date: Apr. 29, 2004

(54) Intraocular lens with photocatalytic coating

(76) Inventors: Ming-Ling Tsai, Taipei (TW); Shen-Wen Cheng, Hsinchu (TW); Tsung-Nan Kuo, Hsinchu (TW); Su-Chen Peng, Taipei (TW)

Correspondence Address: Ladas & Parry, 26 West 61st Street, New York, NY, 10023, US

(21) Appl. No.: 10/431,128

(22) Filed: May 7, 2003

(30) Foreign Application Priority Data
Oct. 25, 2002 (TW) 091125130

(51) Int. Cl. A61F 2/16

(52) U.S. Cl. 623/6.62

(57) ABSTRACT

The subject invention provides an intraocular lens comprising an optic lens body and a layer of a photocatalytic material coated on at least a portion of a surface of the optic lens body. The intraocular lens of the subject invention can eliminate endophthalmitis and inhibit after-cataracts following cataract surgery.

### FIELD OF THE INVENTION

The subject invention relates to an intraocular lens (IOL) on which at least a portion of the lens surface is coated with a photocatalytic material to prevent and control the cataract surgery complications, such as endophthalmitis and after-cataract.

### BACKGROUND OF THE INVENTION

Cataract surgery is one of the most common ophthalmic operations in the world. Standard cataract surgery includes extracapsular lens extraction and intraocular lens implantation. However, several complications, such as endophthalmitis (intraocular infection) and after-cataract (posterior capsular opacity) following cataract surgery, may be occurred after cataract surgery.

Endophthalmitis is a devastating complication of cataract surgery with 0.05% to 0.5% incidence rate; which is characterized by hypopyon and vitreous cavity pus formation. This devastating intraocular infection results from the introduction of microorganism into ocular anterior chamber during cataract surgery. The microorganisms of endophthalmitis commonly include Staphylococci aureus, Pseudomonus aeriginosa, and Escherichia coli. These microorganisms may arise from contaminated instruments or patient's periorbital region, such as eyelash and conjunctiva. The microorganisms existed in ocular anterior chamber may pass through pupil and lens front surface into vitreous cavity after cataract surgery. Once the organisms gain access to the vitreous cavity, severe inflammation may occur and eventually result in severe visual loss. However, Ciulla T A, et al., “Bacterial endophthalmitis prophylaxis for cataract surgery: an evidence-based update,” Ophthalmology, 2002, January;109(1):13-24, reports that there is no effective method or procedure can prevent endophthalmitis such as pre-operative ocular irrigation, intra-operative antibiotic irrigation and post-operative antibiotic injection.

A variety of techniques have been proposed for the prevention and treatment of endophthalmitis. U.S. Pat. No. 5,843,186 discloses an intraocular lens with antimicrobial properties, which involves the use of iontophoretic polymers in fabricating at least a portion of intraocular lens in inhibiting bacteria. Portoles M, et al., “Reduced bacterial adhesion to heparin-surface-modified intraocular lenses,” J. Cataract. Refract. Surg., 1993, November;19(6):755-9, describes that intraocular lens coated with heparin can reduce the adhesion of bacteria on the lens, thus lowering the risk of inflammation and infection. So far, the most effective procedure for the treatment of endophthalmitis is pars plana vitrectomy combined fortified antibiotics irrigation. However, severe visual loss may still eventually occur in patients even though they have been received above aggressive treatments. Therefore, endophthalmitis is a nightmare to ophthalmic surgeons.

After-cataract is the most common complication occurred in 5%-50% of patient following cataract surgery. After-cataract results from migration and proliferation of residual lens epithelial cells on the lens post capsule. Lens epithelial cells on the lens post capsule may result in obstruction of image refractive onto the retina and lead to blurring vision. Currently, a variety of techniques have been proposed for the prevention and treatment of after-cataracts. U.S. Pat. No. 6,248,734 discloses that lens epithelial cells can be inhibited by photosensitizers, such as green porphyrins. Latz C, et al., “Inhibition of lens epithelial cell proliferation by substituted PMMA intraocular lenses,” Graefes. Arch. Clin. Exp. Ophthalmol., 2000, August;238(8):696-700, also describes that lens epithelial cells proliferation can be inhibited by intraocular lens made of COO⁻ and SO₃⁻-substituted acrylic polymer. U.S. Pat. No. 5,273,751 discloses that residual lens epithelial cells after lens extraction can be inhibited by intracameral injection with cell-killing substance. The cell-killing substance is preferably an acid or base adjusted aqueous solution having a pH of ranging from about 1.0 to below 6.5 or about above 7.5 to 14.0. So far, the most efficient technique for the treatment of after-cataract is neodymium yttrium-aluminum-garnet (Nd-YAG) laser capsulotomy. The laser procedure destroys opacity posterior capsule, creates clear visual tract and improves visual acuity. However, Nd-YAG capsulotomy may lead to implanted intraocular lens damage and several complications such as retinal detachment. Thus, it is necessary for ophthalmologists to develop another strategy in the prevention or treatment of after-cataract.

Photocatalysts are a group of photo-excitable materials that can be excited by solar radiation especially ultraviolet (UV) or near UV light and lead to the formation of oxygen-containing free radicals. These radicals may attack, oxidize and destroy organic contamination. Cai R. et al., “Induction of cytotoxicity by photoexcited TiO₂ particles,” Cancer Res., 1992, April. 15;52(8):2346-8, describes that photoexcited TiO₂ particles can oxidize and destroy cervical tumor cells. Ohko Y, et al., “Self-sterilizing and self-cleaning of silicone catheters coated with TiO₂ photocatalyst thin films: a preclinical work,” J. Biomed. Mater. Res., 2001;58(1):97-101, reports that silicone catheters or medical tubes coated with TiO₂-photocatalysts can be sterilized and cleaned by irradiation with low-intensity UV light thus is useful in the protection from catheter-related bacterial infections. U.S. Pat. No. 5,958,154 discloses the contact lens coated with titanium oxide to improve the oxygen permeability of the contact lens, reduce the irradiation of ultraviolet light to eyes and achieve self-sterilizing effect. To our knowledge, none of the patents and references teaches or suggests the use of the photocatalytic materials coated on intraocular lens to eliminate endophthalmitis and prevent after-cataract.

### SUMMARY OF THE INVENTION

An object of the subject invention is to provide an intraocular lens coated with photocatalytic materials to eliminate endophthalmitis and inhibit after-cataracts following cataract surgery.

### BRIEF DESCRIPTION OF THE DRAWINGS

Additional objects and features of the present invention will become more apparent and the invention itself will be best understood from the following Detailed Description of the Invention, when read with reference to the accompanying drawings.

FIG. 1 shows the frontal view of one preferred embodiment of the intraocular lens of the invention.

FIG. 2 shows the sagittal view of one preferred embodiment of the intraocular lens of the invention, on which both surfaces (frontal surface and rear surface) of the optical lens body are coated with photocatalytic materials, respectively.

FIG. 3A shows the diagrammatic sketch of one preferred embodiment of the intraocular lens of the invention in human eye, in which a photocatalytic coating on the frontal surface of the intraocular lens is excited by the UV component of solar radiation through pupil thereby to break down bacteria and eliminate endophthalmitis.

FIG. 3B shows the enlarged plan view illustrating the substances in the space in front of the frontal surface of the intraocular lens in FIG. 3A.

FIG. 4A shows the diagrammatic sketch of one preferred embodiment of the intraocular lens of the invention in human eye, in which a photocatalytic coating on the rear surface of the intraocular lens is excited by the UV component of solar radiation through pupil thereby to break down lens epithelial cells.

FIG. 4B shows the enlarged plan view illustrating the substances in the space in the back of the rear surface of the intraocular lens in FIG. 4A.

FIG. 5 shows a diagrammatic sketch of experimental design that evaluates the effect of preferred embodiment of invention.

FIG. 6 shows the effect of the intraocular lens of the invention on bacteria in a lipid peroxidation test, in which I, II, III and IV represent the concentrations of the MDA of groups A, B, C and D.

FIG. 7 shows the effect of the intraocular lens of the invention on bacteria in a colony-forming assay, in which I, II, III and IV represent the ratio of bacterial colony count of groups A, B, C and D over Mock group, respectively.

FIG. 8 shows the effect of the intraocular lens of the invention on lens epithelial cells in a lipid peroxidation test, in which I, II, III and IV represent the concentrations of the MDA of groups A, B, C and D.

FIG. 9 shows the effect of the intraocular lens of the invention on lens epithelial cells in a viable cell count assay, in which I, II, III and IV represent the ratios of the cell counts of groups A, B, C and D over Mock group, respectively.

### DETAILED DESCRIPTION OF THE INVENTION

The preferred embodiments of the present invention described below relate particularly to an intraocular lens. While the description sets forth various embodiment specific details, it will be appreciated that the description is illustrative only and should not to be construed in any way as limiting the invention. Furthermore, various applications of the invention, and modifications thereto, which may occur to those who are skilled in the art, are also encompassed by the general concepts described below.

It is an aspect of the subject invention to provide an intraocular lens, which comprises an optic lens body and a layer of a photocatalytic material coated on at least a portion of a surface of the optic lens body.

According to the invention, the photocatalytic material coated on the surface of the optic lens body is excited by the ultraviolet (UV) or near UV components of incoming light through pupil and generates free radicals, such as superoxide anion or hydroxyl radical. The free radicals can oxidize the bacteria or residual lens epithelial cells attached onto intraocular lens so as to prevent or control lens-related surgery complications such as endophthalmitis or after-cataracts.

The term “intraocular lens,” as used herein, refers to an intraocular implant that refracts incoming light in the eye. Preferably, the intraocular lens comprises an “optical lens body.” The optical lens body may call the “optic”, “optic zone” or “optical lens portion.” Generally, the primary function of the optical lens body is to focus incoming image upon retina. The optical lens body may be a hard or foldable optic transparent plastic which is commonly made of transparent polymeric materials, such as polymethylmethacrylate (PMMA), silicone polymer or acrylic polymers. Commonly, the outer surface of optic lens body comprises a frontal surface (anterior chamber-facing surface) and a rear surface (capsule-facing surface).

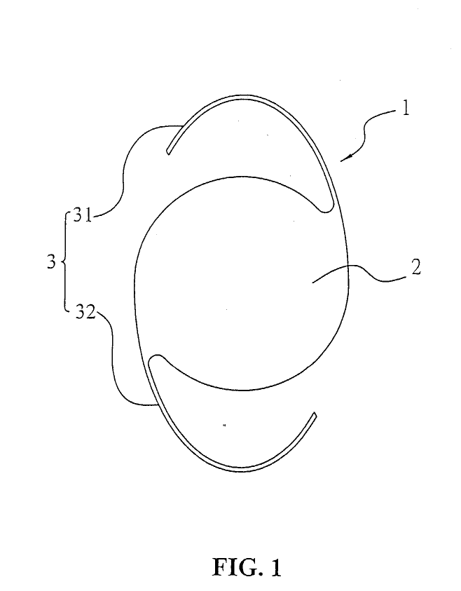

The intraocular lens may further comprise a stabilizing portion used for positioning the optic lens body in alignment with the visual axis of eye. Several designs of the stabilizing portion of the intraocular lens have been reported, such as plate form or filament form consisting of filament-like elements (“haptics”). The haptics are commonly extruded filaments, which commonly made of PMMA, silicone polymer, acrylic polymers, polypropylene or polymerized vinylidene fluoride. Other materials which have been used for fabricating intraocular lens and haptics known in the art are suitable for the subject invention. In one preferred embodiment of the invention, as shown in FIG. 1, the intraocular lens 1 comprises an optical lens body 2 and two filaments-like elements 31 and 32 (haptics 3). The primary function of the optical lens body 2 is to focus incoming light upon the retina. The two filaments-like elements 31 and 32 (haptics 3) can stably position the intraocular lens body 2 in the lens capsular bag 54.

The term “photocatalytic material,” as used herein, refers to a material that can be excited by solar radiation, especially ultraviolet (UV) or near UV light, and lead to the formation of oxygen-containing free radicals. The photocatalytic materials suitable for the subject invention can be any one known to persons skilled in the art. For instance, U.S. Pat. No. 6,290,180 describes a group of photocatalytic materials for semiconductors, such as ZnO, ZnO₃, WO₃, CaTiO₃, SnO₂, MoO₃, KNbO₃, NbO₅, Fe₂O₃, Ta₂O₅, TiO, TiO₂, Ti₂O₃ or Ti₃O₅ or the mixture thereof. In one preferred embodiment of the subject invention, TiO₂ is coated on one surface of the optical lens body to form a layer with a thickness of about 2 nm to about 200 nm to ensure that the TiO₂ layer can be excited by UV irradiation and does not affect the optical properties of the intraocular lens. The excited TiO₂ thus can produce free radicals and attack, oxidize and break down organic contaminants such as bacteria and lens epithelial cells in the eye.

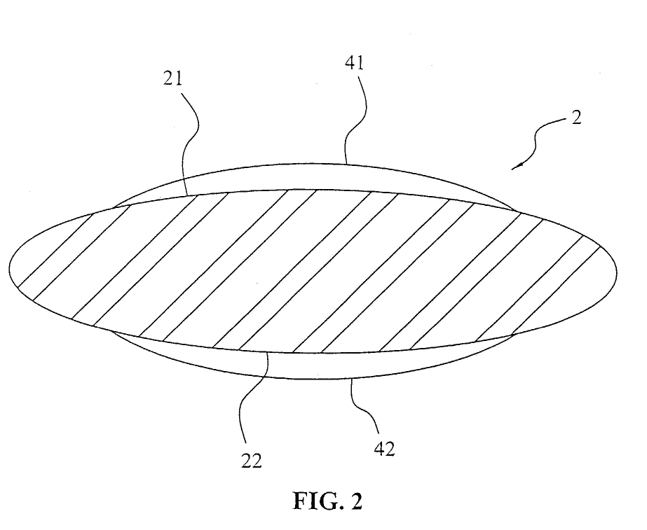

According to the invention, a photocatalytic material is deposited or applied onto at least a portion of a surface of a optical lens body so as to photo-decompose organic contaminants that tend to form on the surfaces of the intraocular lens. The coating of the photocatalytic material onto the intraocular lens can be carried out by using a variety of conventional techniques for forming thin films in the fabrication of integrated circuit, including, but is not limited to sputtering, chemical vapor deposition (CVD), sol-gel method, electron beam physical vapor deposition (EB-PVD), plasma enhanced chemical vapor deposition (PECVD) and physical vapor deposition (PVD). As shown in FIG. 2, in one preferred embodiment of the subject invention, both the frontal surface 21 and the rear surface 22 of the optical lens body 2 lens are coated with a layer of the photocatalytic materials 41 and 42, respectively.

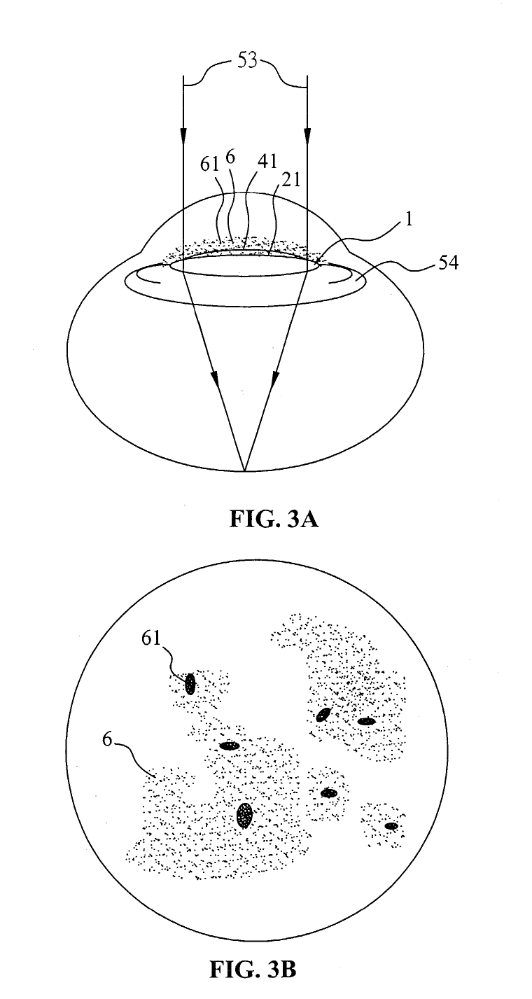

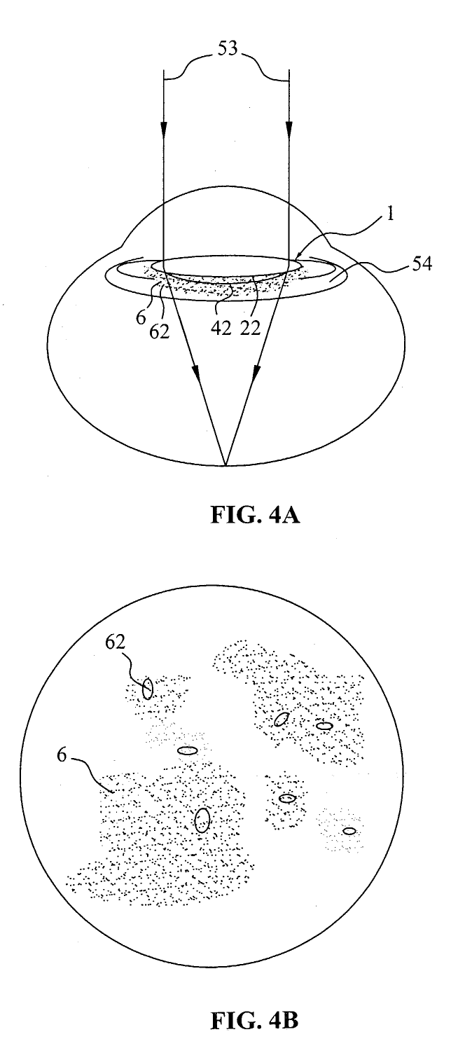

In one preferred embodiment of the invention, as shown in FIG. 3A and FIG. 3B, a layer of the photocatalytic material 41 is coated on a frontal surface 21 (anterior chamber-facing surface) of the optical lens body 2. Thereafter, the intraocular lens of the subject invention was implanted into the lens capsular bag 54 of human eye. According to the subject invention, the photocatalytic coating 41 can be excited by the ultraviolet component 53 of solar radiation through pupil and generate free radicals 6. The free radicals 6 break down bacteria 61 in front of the space of or attached onto the frontal surface, and thus inhibit endophthalmitis. In another preferred embodiment of the invention, as shown in FIG. 4A and FIG. 4B, a layer of the photocatalytic material 42 can also be coated on a rear surface 22 (capsule-facing surface) of the optical lens body 2. The photocatalytic material 42 can be excited by the ultraviolet component 53 of solar radiation through pupil and generate free radicals 6. The free radicals 6 break down residual lens epithelial cells 62 in back of the space of or attached onto the rear surface, and thus inhibit after-cataract. Furthermore, the free radicals exist within nanosecond and move within nanometer. Therefore, according to the subject invention, the photocatalytic material coated on the optical lens body can just oxidize the cells onto the lens surface, which does not compromise the ocular function.

The following examples are for further illustration of the invention but not intended to limit the invention. Any modifications and applications by persons skilled in the art in accordance with the teachings of the invention should be within the scope of this invention.

### EXAMPLE

### Example 1

### Preparation of an Intraocular Lens with a Photocatalytic Coating

Electron beam evaporation of trititanium pentoxide was utilized to deposit a photocatalytic coating onto an intraocular lens that made of silicone polymers. The intraocular lens was placed in reactor at a temperature of 200 to 300° C. under a pressure of 1×10⁻⁴ to 5×10⁻⁴ Torr. The trititanium pentoxide was placed in the tantalum crucible of reactor and evaporated by an electron beam of 8 KeV, 0.2A(1600W) efficiency. Oxygen was supplied to the reactor at a partial pressure in a range of 5 to 10×10⁻⁵ Torr. The trititanium pentoxide was then mostly evaporated into titanium dioxide and deposited onto the lens surface with a thickness of 100 nm. The thickness of the photocatalytic coating on intraocular lens was monitored by an ellipsometer.

The effect of the photocatalytic coating on the intraocular lens on killing microorganisms and lens epithelial cells was evaluated by the following experiments.

### Example 2

### Culture of Microorganisms

E. coli strain ATCC 27325 was cultured in 100 ml of Luria-Bertani broth at 30° C. on a rotary shaker (200 rpm) for 18 hours. The bacterial cells were harvested by centrifugation at 7,800×g for 15 min, washed, and suspended with sterile deionized water to obtain a suspension. The bacteria density of the suspension at 660 nm was determined by measuring the turbidity with a spectrophotometer (Milton Roy Co.).

### Example 3

### The Effect of the Intraocular Lens with a Photocatalytic Coating on Bacteria

The effect of the intraocular lens with a photocatalytic coating on killing bacteria was evaluated by a lipid peroxidation test (MDA-TBA assay) and a bacterial colony-forming assay.

Pre-Treatment of Bacteria Suspension

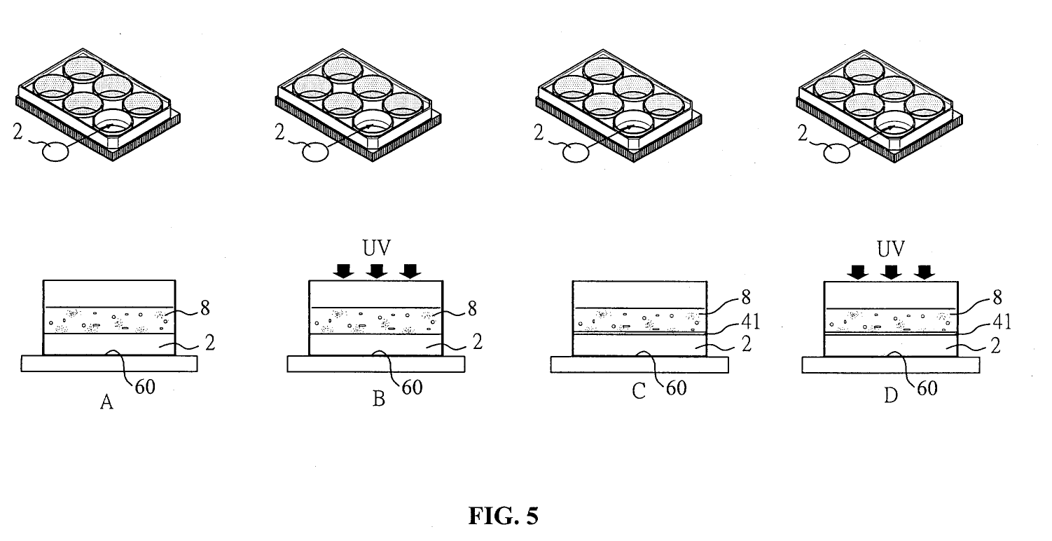

As shown in FIG. 5, different groups (A, B, C, D and Mock) of culture plates were used for the lipid peroxidation test and bacterial colony-forming assay. Intraocular lens bodies 2 (6 mm in diameter) with or without a photocatalytic coating were then placed on the wall bottom 60 (6 mm in diameter) of each well in the different groups of culture plates. Intraocular lens body 2 with a photocatalytic coating 41 was placed in the culture plate groups C and D, respectively; intraocular lens body 2 without a photocatalytic coating was placed in another culture plate groups A and B, respectively. Intraocular lens body 2 without a photocatalytic coating was placed in another culture well plates as Mock group. A 500 μl suspension of E. coli cells with a density of 1×10⁵ cells/mL were then placed and cultured in each culture well plate of different groups. The cells in plate groups B and D were cultured in an incubator with UV light illumination (type F40/BL-B; Sylvania). The UV light intensity reaching the surface at the center of well plates was adjusted to approximately 8 Wm⁻² by a long-wavelength UV meter (model J-221 long-wavelength UV meter; UVP Inc., San Gabriel, Calif.). The cells in plate groups A, C and Mock were cultured without UV light illumination. After one hour, the cell suspension 8 of each plate groups A (N=12), B (N=12), C (N=12), D (N=12) and Mock group (N=12) was used for the following tests.

Lipid Peroxidation (MDA-TBA Assay)

The oxidation capacity of the intraocular lens of the invention on bacteria was determined by lipid peroxidation. The formation of malondialdehyde (MDA) was used as an index to measure lipid peroxidation, because MDA was released after bacteria had been oxidized. The MDA was quantified based on its reaction with trichloroacetic acid (TBA) to form a pink MDA-TBA adduct.

The bacterial cell suspension was collected in each culture well of different group and mixed with two volume of 10% (wt/vol) TBA, and the solids were removed by centrifugation at 11,000×g for 35 minutes and then for an additional 20 minutes to ensure that the bacterial cells and precipitated proteins were completely removed. Three volume of a freshly prepared 0.67% (wt/vol) TBA (Sigma Chemical Co.) solution was then added to the supernatant. The samples were incubated in a boiling-water bath for 10 minutes and then cooled, and measured with the absorbance at 532 nm by a Cary 5E spectrophotometer (Varian Instruments, Sugar Lane, Tex.). The concentrations of the MDA formed were calculated based on a standard curve for the MDA (Sigma Chemical Co.) complex with TBA.

Bacterial Colony-Forming Assay

The bacterial survival rates of different groups were evaluated by colony-forming assay to investigate the effect of the intraocular lens of the subject invention on bacteria. The bacterial cell suspension after treatment in each culture well of different group was collected and plated onto different Luria-Bertani agar plates. The agar plates were incubated at 37° C. for 24 hours, and then the numbers of colonies on the plates of each group were counted. The ratios of colony counts of groups A, B, C and D over Mock group were calculated respectively as the results of the bacteria survival rate (see I, II, III and IV in FIG. 7).

Results

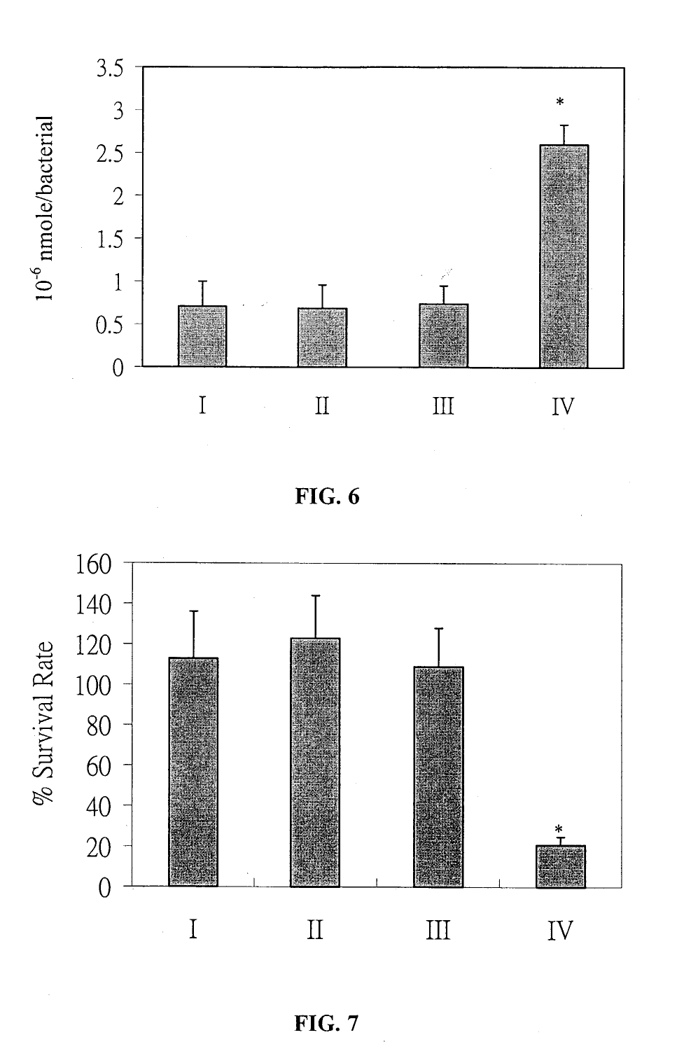

As shown in FIG. 6 and FIG. 7, we observed that intraocular lens with a photocatalytic coating under UV irradiation break down E. coli cells. The results reveal that the intraocular lens of the invention significantly destroys bacteria (ANOVA; P value<0.01) and thus eliminates endophthalmitis.

### Example 4

### Culture of Lens Epithelial Cells

Human lens epithelial cells (ATCC CRL-11421; B3 cells) were cultured in an Eagles minimum essential medium containing 10% fetal calf serum, penicillin 100U/ml, and streptomycin sulfate (100 ug/ml). The specimen was cultured in 100% humidity at 37° C. in a 5% carbon dioxide atmosphere. The culture medium was replaced every 7 days. Cell growth in each culture plate was observed daily under an inverted phase-contrast microscope.

### Example 5

### The Effect of Intraocular Lens with Photocatalytic Coating on Lens Epithelial Cells

The effect of intraocular lens with a photocatalytic coating on lens epithelial cells was evaluated by an MDA-TBA assay and a viable cell count assay.

Pre-Treatment of Lens Epithelial Cells

The pre-treatment of the intraocular lens was performed in accordance with the procedures described in Example 3, except that lens epithelial cell suspensions were used for substitution of the bacterial cell suspensions.

Lipid Peroxidation Test

The MDA-TBA assay was performed in accordance with the procedures described in Example 3, except that lens epithelial cell suspensions were used for substitution of the bacterial cell suspensions.

Viable Cell Count Assay

The viable cell count assay was performed in accordance with the procedures of the bacterial colony-forming assay described in Example 3, except that lens epithelial cell suspensions were used for substitution of the bacterial cell and incubated for 6 hours. After incubation, the lens body of intraocular lens was removed from culture wells, fixed with 70% methanol, stained with a 5% Giemsa solution, and the numbers of the viable lens epithelial cell were counted. The ratio of colony viable cell counts of groups A, B, C and D over Mock group was calculated respectively as the results of the cell survival rate (see I, II, III and IV in FIG. 7).

Results

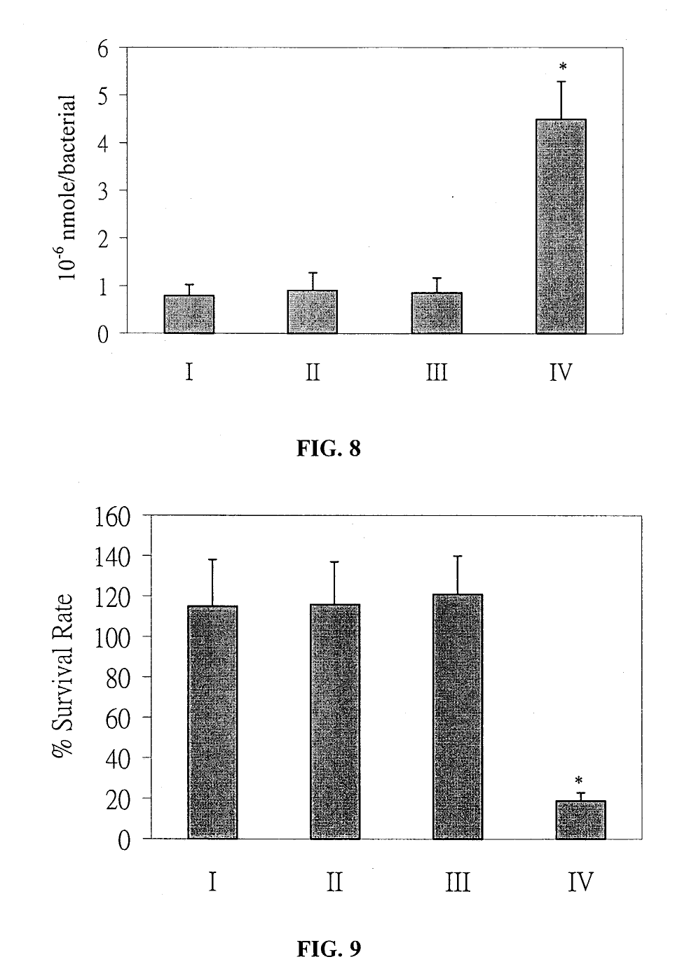

As shown in FIG. 8 and FIG. 9, intraocular lens with a photocatalytic coating under UV irradiation break down lens epithelial cells. The results reveal that the intraocular lens of the invention significantly breaks down lens epithelial cells (ANOVA; p<0.01) and thus inhibit after-cataract.

We claim:

1. An intraocular lens comprising an optic lens body and a layer of a photocatalytic material coated on at least a portion of a surface of the optic lens body.

2. The intraocular lens according to claim 1, wherein the optic lens body is made of polymethylmethacrylate (PMMA), silicone polymer or acrylic polymer.

3. The intraocular lens according to claim 1, wherein the photocatalytic material is selected from the group consisting of ZnO, ZnO₃, WO₃, CaTiO₃, SnO₂, MoO₃, KNbO₃, NbO₅, Fe₂O₃, Ta₂O₅, TiO, TiO₂, Ti₂O₃ or Ti₃O₅ or the mixture thereof.

4. The intraocular lens according to claim 3, wherein the photocatalytic material is TiO₂.

5. The intraocular lens according to claim 1, wherein the layer of a photocatalytic material has a thickness of about 2 nm to about 200 nm.

6. The intraocular lens according to claim 1, wherein the surface of the optic lens body is a frontal surface (anterior chamber-facing surface) of the optical lens body.

7. The intraocular lens according to claim 1, wherein the surface of the optic lens body is a rear surface (capsule-facing surface) of the optical lens body.

8. The intraocular lens according to claim 1, further comprising two filament-like elements.

9. The intraocular lens according to claim 8, wherein the filament-like elements are made of PMMA, silicone polymer, acrylic polymers, polypropylene or polymerized vinylidene fluoride.
